(19) United States
(12) Patent Application Publication
(10) Pub. No.: US 2024/0049671 A1
(43) Pub. Date: Feb. 15, 2024

(54) HEXAPLOID-OCTOPLOID PERENNIAL RYEGRASS, HEXAPLOID-OCTOPLOID RYEGRASS AMERICAN RYEGRASS, AND HYBRIDS THEREOF

(71) Applicant: NEXGEN Plant Science Center, LLC, Albany, OR (US)

(72) Inventors: Kenneth Hignight, Albany, OR (US); Debra Hignight, Albany, OR (US); Joseph K. Wipff, Albany, OR (US); Miles A. Barrett, Albany, OR (US)

(21) Appl. No.: 18/232,269

(22) Filed: Aug. 9, 2023

Related U.S. Application Data
(60) Provisional application No. 63/396,497, filed on Aug. 9, 2022.

(51) Int. Cl. A01H 6/46; A01H 1/08; A01H 5/10

(52) U.S. Cl. CPC A01H 6/46; A01H 1/08; A01H 5/10

(57) ABSTRACT

A method of developing hexaploid (2n=6x=42) and octoploid (2n=8x=56) turf-type perennial ryegrass (Lolium perenne L.) and Festulolium [(American Ryegrass), x Festulolium loliaceum (Huds.) P. Fourn. (Festuca pratensis Huds. x Lolium perenne)], and their hybrids with Tall Fescue (Festuca arundinacea Schreb.) including germplasm, cultivars and seed used to produce the grass are provided. These grasses are useful as an improved turfgrass on golf courses, athletic fields and other areas using seeded turfgrasses. The grasses will provide a green improved winter performance and drought tolerance.

### CROSS-REFERENCE TO RELATED APPLICATION(S)

This application claims the benefit under 35 U.S.C. § 119(e) to U.S. Provisional Application 63/396,497, filed Aug. 9, 2022, and entitled TURFGRASS: HEXAPLOID-OCTOPLOID PERENNIAL RYEGRASS; HEXAPLOID-OCTOPLOID RYEGRASS AMERICAN RYEGRASS AND THEIR HYBRIDS, which is hereby incorporated herein by reference in its entirety for all purposes.

### TECHNICAL FIELD

The disclosure relates to cultivated varieties of grass and their hybrids, and methods for their cultivation and breeding.

### BACKGROUND

The ploidy levels of the worldwide perennial ryegrass accessions were screened in the USDA National Plant Germplasm System (NPGS). Among the 200 accessions screened, 194 diploids (2n=2x=14) and six tetraploids (2n=4x=28) were identified. They did not find any hexaploid (2n=6x=42) nor octoploid (2n=8x=56) accessions of perennial ryegrass.

The development of tetraploid (2n=4x=28) perennial ryegrass cultivars from diploid (2n=2x=14) germplasm is described in previous art developed and patented by NexGen Plant Science Center (U.S. Pat. No. 7,368,638 incorporated herein by reference).

Festulolium is the name for intergeneric/interspecific hybrid grasses developed by crossing species of Festuca and Lolium. Festulolium refers to natural or synthetic intergeneric hybrids between obligate outbreeding species of the Festuca (fescue) and Lolium (ryegrass) genera, species considered frequently as components of agricultural or turf-grass systems. Intermediate forms between the two genera have long been recognized in nature and considered as hybrids (x Festulolium spp.) by taxonomists mostly on the basis of the inflorescence shape and their suspected progenitor species' combinations. The development of a Festulolium turfgrass is described in previous art developed and patented by NexGen Plant Science Center (U.S. Pat. No. 10,721,879).

Historically and traditionally, Festulolium cultivars have only been developed for forage qualities (Ghesquiere et al., 2010; Hopkins et al., 2009). American ryegrass, was the first and previously only Festulolium turfgrass [x Festulolium loliaceum (Huds.) P. Fourn. (Festuca pratensis Huds. x Lolium perenne)] developed and is described as previous art developed and patented by NexGen Plant Science Center (U.S. Pat. No. 10,721,879 B2).

There is a need in the art for varieties of hexaploid (2n=6x=42) and octoploid (2n=8x=56) perennial ryegrass, and/or American ryegrass (turf-type Festulolium), and their hybrids with tall fescue.

### BRIEF SUMMARY

Disclosed herein are various cultivated varieties of true breeding, stable, hexaploid (2n=6x=42) and octoploid (2n=8x=56) germplasm of perennial ryegrass and American ryegrass (turf-type Festulolium, described in U.S. Pat. No. 10,721,879 incorporated herein by reference), and their hybrids with tall fescue (Festuca arundinacea Schreb.). The various varieties described herein may be for use as a short lived, improved turfgrass on golf courses, athletic fields and other areas using seeded turfgrasses. The grasses will provide a green improved winter performance and drought tolerance.
In Example 1 a seed of a hexaploid perennial ryegrass (breeders code AHPR001), optionally Lolium perenne.
Example 2 relates to a grass plant produced by growing the seed of Example 1.
Example 3 relates to a grass plant having all the physiological and morphological characteristics of the grass plant of Example 2.
In Example 4 a seed of an octoploid perennial ryegrass (AOPR001), optionally Lolium perenne.
Example 5 relates to a grass plant produced by growing the seed of Example 4.
Example 6 relates to a grass plant having all the physiological and morphological characteristics of the grass plant of Example 5.
In Example 7 a seed of a hexaploid American ryegrass (AHPM001), optionally Festulolium loliaceum.
Example 8 relates to a grass plant produced by growing the seed of Example 7.
Example 9 relates to a grass plant having all the physiological and morphological characteristics of the grass plant of Example 8.
In Example 10 a seed of an octoploid American ryegrass (AOPM001), optionally Festulolium loliaceum.
Example 11 relates to a grass plant produced by growing the seed of Example 10.
Example 12 relates to a grass plant having all the physiological and morphological characteristics of the grass plant of Example 11.
In Example 13 a seed of a hexaploid hybrid of the seed of Example 1 (optionally hexaploidy Lolium perenne) and Festuca arundinacea (Tall Fescue) (ATFP001), optionally x Festulolium holmbergii.
Example 14 relates to a grass plant produced by growing the seed of Example 13.
Example 15 relates to a grass plant having all the physiological and morphological characteristics of the grass plant of Example 14.
In Example 16 a seed of an hexaploid hybrid of the seed of Example 7 (hexaploid Festulolium loliaceum) and Festuca arundinacea (Tall Fescue) (ATFF001), optionally x Festulolium sp.
Example 17 relates to a grass plant produced by growing the seed of Example 16.
Example 18 relates to a grass plant having all the physiological and morphological characteristics of the grass plant of Example 17.
In Example 19 a seed of a hexaploid hybrid of the seed of claim 13 (x Festulolium holmbergii) and the seed of claim 16 (x Festulolium sp.) (AHFF001).
Example 20 relates to a grass plant produced by growing the seed of claim 19.
Example 21 relates to a grass plant having all the physiological and morphological characteristics of the grass plant of claim 20.

While multiple embodiments are disclosed, still other embodiments of the disclosure will become apparent to those skilled in the art from the following detailed description, which shows and describes illustrative embodiments of the invention. As will be realized, the disclosure is capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 is a flow diagram showing hybridization and breeding of the various cultivars described herein, according to one implementation.

### DETAILED DESCRIPTION

Stable varieties of hexaploid (2n=6x=42) and octoploid (2n=8x=56) perennial ryegrass, and hexaploid (2n=6x=42) and octoploid (2n=8x=56) American ryegrass (turf-type Festulolium), and their hybrids with tall fescue are a desirable grass for turf cover during the cool winter months and through the summer. Turfgrass managers may utilize the disclosed cultivars that have a dark green color, rapid establishment, and with good winter color and drought tolerance. Hexaploid (2n=6x=42) and octoploid (2n=8x=56) perennial ryegrass, and hexaploid (2n=6x=42) and octoploid (2n=8x=56) American ryegrass (turf-type Festulolium), and their hybrids with Tall Fescue (Festuca arundinacea Schreb.), as will be described herein, have not been previously developed or released for turfgrass use.

Herein turf type cultivars have been developed and are described specifically for turf applications from hexaploid (2n=6x=42) and octoploid (2n=8x=56) perennial ryegrass, and hexaploid (2n=6x=42) and octoploid (2n=8x=56) American ryegrass (turf-type Festulolium), and their hybrids with tall fescue (Festuca arundinacea Schreb.).

Octoploid (2n=8x=56) perennial ryegrass is made by doubling the chromosomes of a tetraploid (2n=4x=28) perennial ryegrass. The hybridization of an octoploid (2n=8x=56) and tetraploid (2n=4x−28) ryegrass results in hexaploid (2n=6x=42) progeny. These two ploidy levels are developed into turfgrass varieties as is described herein.

Octoploid (2n=8x=56) Festulolium is made by doubling the chromosomes of a tetraploid (2n=4x=28) Festulolium. The hybridization of an octoploid (2n=8x=56) and tetraploid (2n=4x=28) Festulolium results in hexaploid (2n=6x=42) progeny. These two ploidy levels are developed into turfgrass varieties as is described herein.

Also described herein is x Festulolium holmbergii (Dörfl.) P. Fourn. [Festuca arundinacea x Lolium perenne] the second species of turf Festulolium to be developed. Hexaploid (2n=6x=42) perennial ryegrass is crossed with hexaploid (2n=6x=42) tall fescue to produce a new turfgrass species called x Festulolium holmbergii (Dörfl.) P. Fourn. (Festuca arundinacea x Lolium perenne) with breeders code ATFP001. Further, the crossing the hexaploid (2n=6x=42) Festulolium with hexaploid (2n=6x=42) Tall Fescue [Festuca arundinacea x x Festulolium loliaceum] to produce a new type of Festulolium hybrid with breeders code: APMF001 and ATFF001. A further cross of x Festulolium holmbergii with the seed of the hybrid of [Festuca arundinacea x x Festulolium loliaceum] is used to create a further species, with breeders code AHFF001.

Aneuploid is the occurrence of one or more extra or missing chromosomes leading to an unbalanced chromosome complement, or any chromosome number that is not an exact multiple of the haploid number or base chromosome number of the organism being studied.

Base chromosome number represents the lowest known ancestral chromosome number in the haploid complement of a species and is designated by letter ‘x’. In angiosperms, the base number ranges from as low as x=2 in Haplopappus to as high as x=43 in some species of family Winteraceae.

Colchicine is a mutagen that works by preventing the microtubules formation and doubles the number of chromosomes. It is commonly used to develop polyploid plants and functions as a mitotic poison by producing many mutagenic effects on plants.

A Diploid is a cell that contains two complete sets of chromosomes. This is double the haploid chromosome number. Each pair of chromosomes in a diploid cell is considered to be a homologous chromosome set. The total number of chromosomes in diploid cells is described as 2n, which is twice the number of chromosomes in a haploid cell (n). This could be written 2n=2x=chromosome number. 2n is he somatic (sporophyte) cell number designation for plants that are diploid or have polyploid chromosome number(s).

A Haploid has a single set of unpaired chromosomes.

A Hexaploid contains six homologous sets of chromosomes. Written 2n=6x=chromosome number.

A Octoploid contains eight homologous sets of chromosomes. Written 2n=8x=chromosome number.

A Polyploid or Polyploidy is a condition in which the cells of an organism have more than one pair of (homologous) chromosomes.

A Tetraploid contains four homologous sets of chromosomes. Written 2n=4x=chromosome number.

Turf type APMT hexaploid (2n=6x=42) and octoploid (8x) Festulolium, and hexaploid (2n=6x=42) and octoploid (2n=8x=56) APRT perennial ryegrass cultivars known as breeder's codes: AHPM001 (hexaploid Festulolium (American Ryegrass)); AOPM001 (octoploid Festulolium (American Ryegrass)); AHPM001 (hexaploidy perennial ryegrass); and AOPR001 (octoploid perennial ryegrass), and methods used to produce the grass are provided.

Turf type interspecific hybrids resulting from the hybridization of hexaploid (2n=6x=42) Festulolium and hexaploid (2n=6x=42) Tall Fescue (including, but not exclusive to) known as breeder's codes: ATFF 001, and methods used to produce the grass are provided.

Turf type interspecific hybrids resulting from the hybridization of 5 exaploidy (2n=6x=42) Perennial Ryegrass and 5 exaploidy (2n=6x=42) Tall Fescue (including, but not exclusive to) known as breeder's codes: APTF 001, and methods used to produce the grass are provided.

The various grasses described herein are useful as improved turfgrass on golf courses, athletic fields, lawns, and other areas using seeded turfgrasses. The grass is also useful in overseeding of cool season grasses. Turf type 5 exaploidy (2n=6x=42) and octoploid (2n=8x=56) Festulolium, turf type 5 exaploidy (2n=6x=42) perennial ryegrass, and their hybrids from crosses with 5 exaploidy (2n=6x=42) Tall Fescue, in more temperate climates, will provide a permanent turf more cold tolerance, with better winter color than Tall Fescue, and better drought tolerance and persistence than diploid (2n=2x=14) perennial ryegrass. Turf type 5 exaploidy (2n=6x=42) and octoploid (2n=8x=56) Festulolium, turf type 5 exaploidy (2n=6x=42) and octoploid (2n=8x=56) perennial ryegrass and turf type interspecific hybrids resulting from the hybridization of 5 exaploidy (2n=6x=42) Festulolium and 5 exaploidy (2n=6x=42) Tall Fescue have dark green color and rapid establishment that could be utilized by turfgrass managers.

As used herein the term establishment indication the ability of a seed to germinate and tiller into areas adjacent to the seedling. It is important in an overseeding grass to have rapid establishment. This includes a quick germination of the seed and the ability to tiller into areas adjacent to the next seedling.

As used herein the term color refers to the color of the leaves/blades of the plant. Many users of overseeding grasses prefer a dark green color. A light color grass can be made darker by applying iron. However, the user generally prefers to not do this unless the grass is too light in color.

As used herein the term transition refers to the ability of a grass to die as the seasons change. A desirable transition grass is one that will die completely when the warm season grass is starting to reach its peak performance. The overseeding grass cannot die too quickly in the spring before the warm-season grass has an opportunity to grow.

As used herein the term turf describes a covering of mowed vegetation usually a grass. Turfgrass is a species or cultivar of grass that is a mowed turf.

### Species

Annual ryegrass—Annual ryegrass was used extensively for overseeding prior to 1970. With breeding improvements of perennial ryegrass, the use of annual ryegrass has declined. Annual ryegrass has an excellent germination rate but lacks tillering ability. Annual ryegrass is light in color and usually transitions too abruptly. The course leaf texture and very fast growth rate are undesirable.

Diploid Perennial ryegrass—Perennial ryegrass has a quick germination. It germinates slower than annual ryegrass but still within an acceptable range for the user. It has a very good tillering ability but, in some cases, it is too aggressive resulting in damage to the warm season grass. Newer cultivars of perennial ryegrass have excellent, dark green, color. The newer varieties of perennial ryegrass which are being sold as overseeding grasses were developed for permanent turf use. The result is a poor transitioning ability. This has resulted in the use of chemical applications to remove the perennial ryegrass. When this is necessary the turf has damage for several weeks, until the warm-season grass can recover.

Intermediate ryegrass— Intermediate ryegrass performance is more difficult to predict. It is a cross between annual and perennial ryegrass. If only one cross is made the performance is most similar to annual ryegrass. Each successive backcross to perennial ryegrass results in performance similar to diploid perennial ryegrass. A problem with current intermediate ryegrass is that it often transitions too rapidly, before the warm-season grass has a desirable level of performance.

Poa trivialis and Fine fescues—Both of these have slow germination and establishment. Because of this they are commonly used in mixtures with other grasses. The cost of producing these grasses is higher and as a result they are not usually used alone. The fine fescues have good transitioning ability compared to Poa trivialis.

American Ryegrass—American Ryegrass has a rich very dark green color, high tiller density and slow vertical growth rate. Previous Festulolium cultivars have been developed for forage use, and therefore have a light color, poor turf density, and a rapid vertical growth rate. American Ryegrass has a rapid germination and establishment rate, and transitions when used in overseeding with Bermuda grass (warm-season). American Ryegrass transitions better than diploid perennial ryegrass. American Ryegrass in more temperate climates, will provide a permanent turf more cold tolerance, with good winter color, and better drought tolerance and persistence than perennial ryegrass.

Tall Fescue—Tall Fescue is valued for its adaptability to a wide range of climates and its tolerances for cold, heat, drought, and shade. As a cool-season grass, Tall Fescue is well-suited to northern lawns. But it also has added value in southern transitional turf grass regions where cool-season and warm-season grasses meet their climate limits. Tall Fescue offers greater heat tolerance than other cool-season grasses and greater cold tolerance than warm-season grass options for these lawn owners. The result is beautiful year-round lawns in this challenging transition area.

Tetraploid Perennial Ryegrass—Tetraploid perennial ryegrass has a rich very dark green color, high tiller density and slow vertical growth rate. Most tetraploid ryegrasses have been developed for forage use and therefore have a light color, poor turf density, and a rapid vertical growth rate. Tetraploid perennial ryegrass has a rapid germination rate and establishment rate and transitions when used in overseeding with Bermuda grass (warm-season). Tetraploid ryegrass transitions better than diploid perennial ryegrass.

### Chromosome Doubling

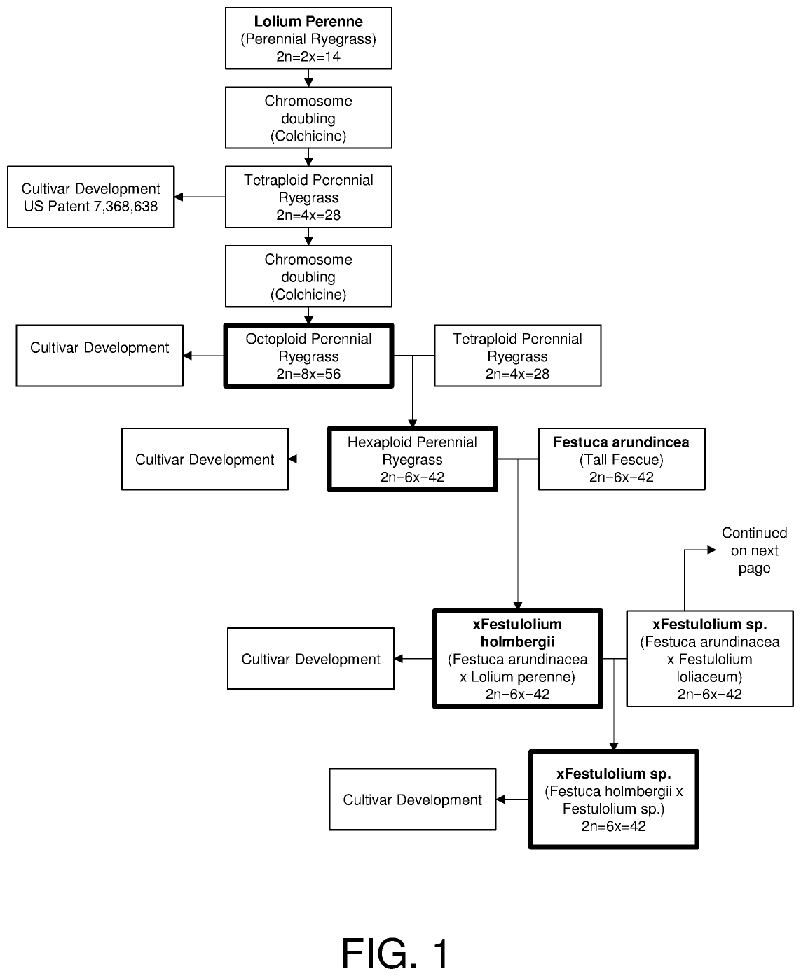

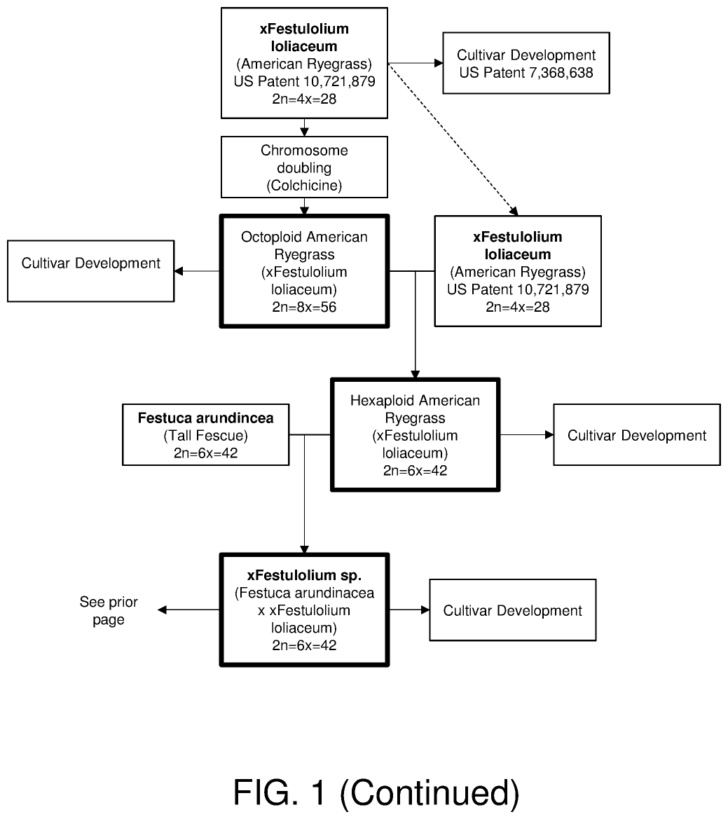

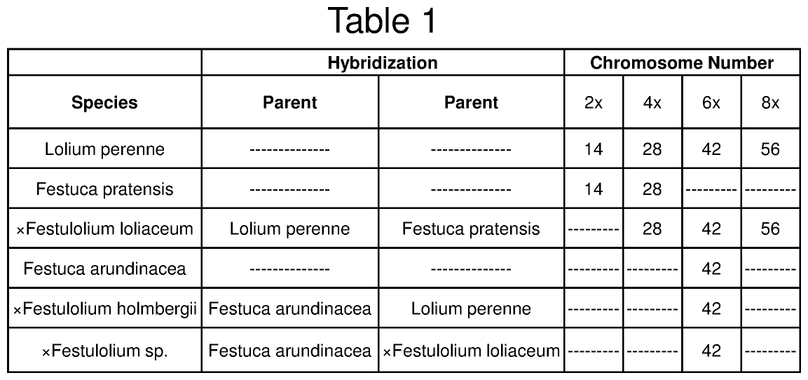

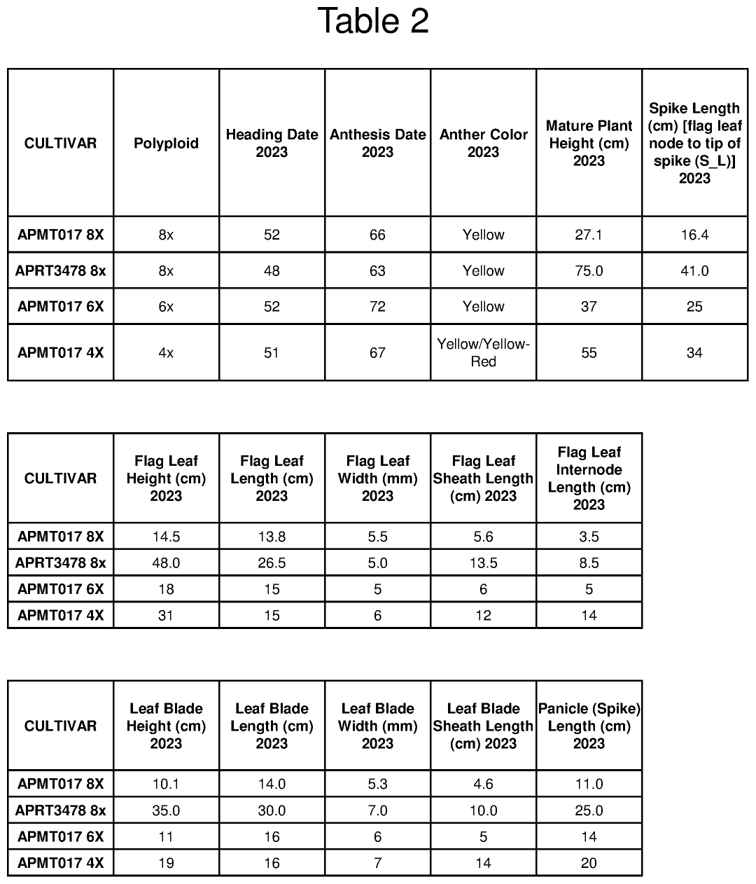

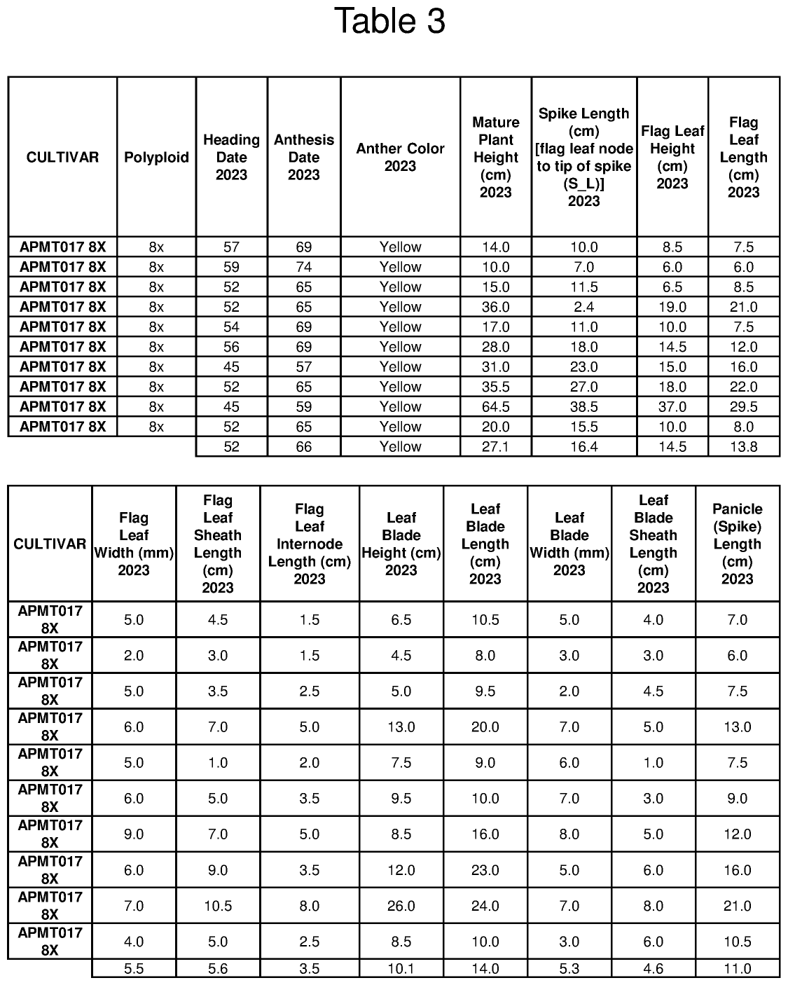

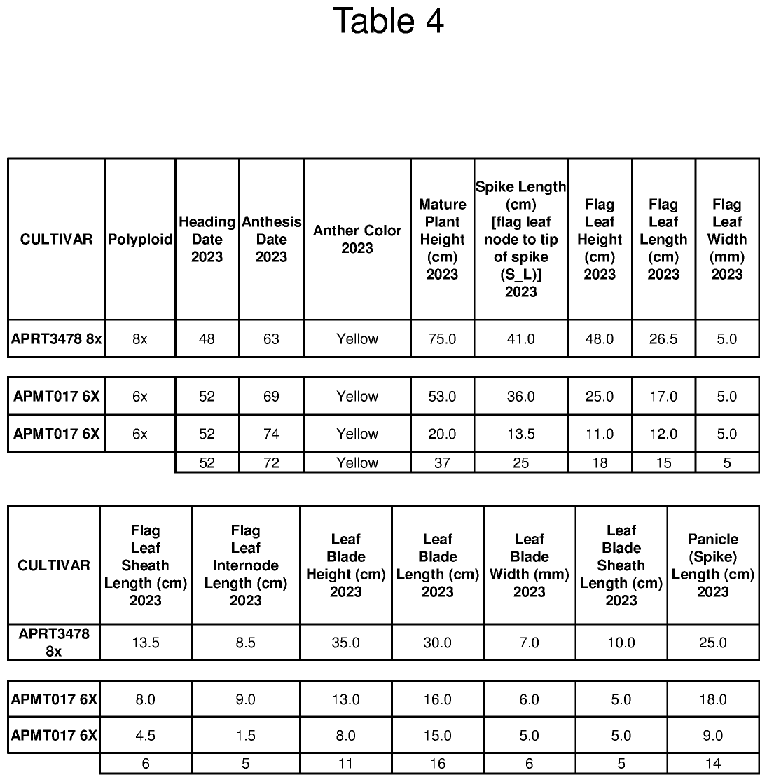

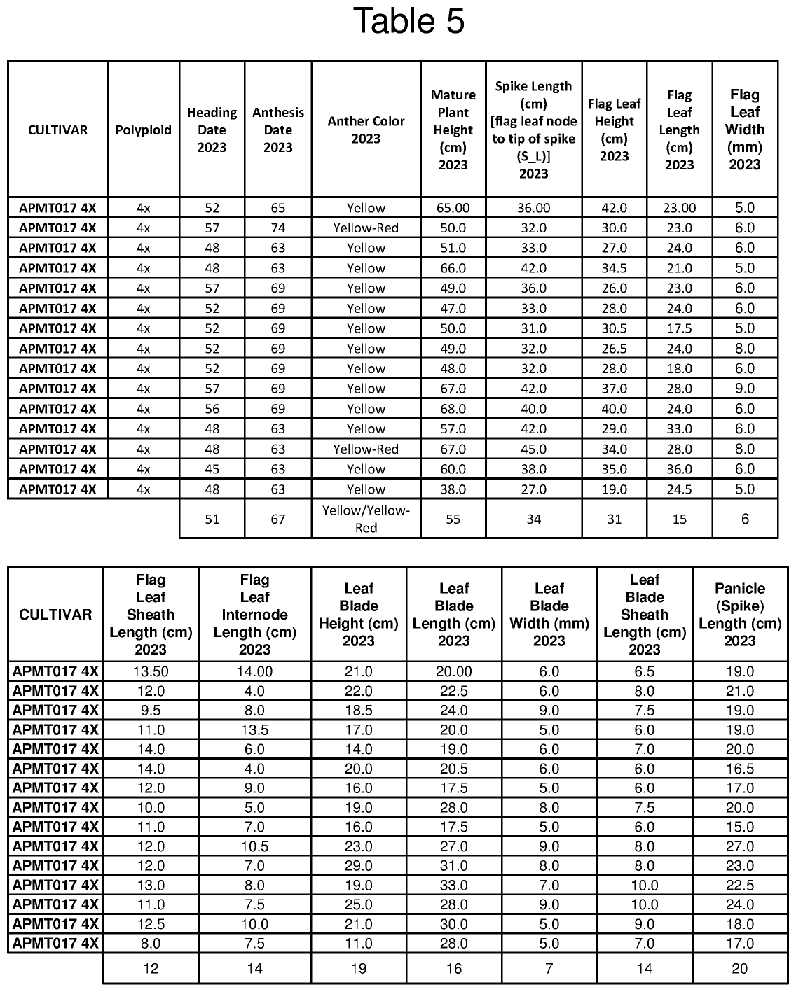

A flow diagram showing the chromosome doubling is shown in FIG. 1.

In order to doubling the chromosome number in plants, 2n=2x=14 plants are doubled to 2n=4x=28 plants. Then 2n=4x=28 plants are doubled to 2n=6x=56 plants.

In one example, the seeds are sprinkled on a 6×9-inch wet blotting paper in a 9½″×6 5/16″×1½″ OD acrylic container used for seed germination. A lid is placed on the container and placed in a germinator for 3-4 days in a controlled environment: 21° C. and 12 hours of light, until seeds begin germinating.

The seeds are then inspected for root length. The root length for colchicine treatment is 2-3 mm. Then, all the seeds with root length of 2-3 mm are placed in a petri dish. During this process the seeds are kept separate by experimental line.

A Colchicine solution: 0.2 grams colchicine, 1 ml Tween 80, 1 ml DMSO, 98 ml distilled water is then prepared. Three milliliters of colchicine solution is added to each Petri dish and a 3 ml of distilled water is added to another Petri dish which will act as an untreated check of each experimental line.

The Petri dishes are then shaken gently for good distribution of the colchicine. The colchicine treated seeds are placed in the dark for 2 hours at room temperature 30° C.

The seeds are then placed in a strainer and rinsed with copious amounts of water for 15 minutes. The seeds are placed on blotting paper and placed in a dark cabinet for 5 days.

For many of the seeds, the colchicine will be lethal; for some of the seeds the colchicine will not enter the tissue, resulting in normal 2n=2x=14 plants.

The 2n=4x=28 and 2n=8x=56 plants can be removed from the other seedlings. The 2n=4x=28 and 2n=8x=56 plants are thicker. The 2n=4x=28 and 2n=8x=56 plants are planted in soil and moved to the greenhouse. The 2n=4x=28 and 2n=8x=56 plants are confirmed by flow cytometry. Anything confirmed to be 2n=4x=28 and 2n=8x=56 is moved into a larger pot for holding. Anything confirmed to be 2n=2x=14 or Aneuploid, is discarded.

The 2n=4x=28 and 2n=8x=56 plants are moved into isolated crossing block in field. In the spring, plants in the field are rechecked, by flow cytometry, to confirm all are still 2n=4x=28 and 2n=8x=56. Any plants not 2n=4x=28 and 2n=8x=56 are removed from the nursery. In the summer plants are harvested and seed is cleaned and prepared.

### Development of Hexaploid Chromosome Numbers and Hybridizations

A table showing the hybridizations is shown in Table 1 and diagramed in the flow diagram of FIG. 1.

The development of 7 exaploidy (2n=6x=42) cultivars is done through hybridization of 2n=4x=28 and 2n=8x=56 polyploid plants of perennial ryegrass and Festulolium.

In one such implementations, 2n=4x=28 perennial ryegrass is crossed with 2n=8x=56 perennial ryegrass to produce progeny that are 7 exaploidy (2n=6x=42) and fertile.

In another implementation, 2n=4x=28 Festulolium is crossed with 2n=8x=56 Festulolium to produce progeny that are 7 exaploidy (2n=6x=42) and fertile.

These 7 exaploidy (2n=6x=42) progeny are then used to develop improved turfgrass cultivars and are used in further hybridizations to develop new turfgrass cultivars, described herein.

### Cultivar Development

In various implementations, turfgrass cultivars are developed from the 2n=4x=28 and 2n=8x=56 perennial ryegrass and 2n=8x=56 Festulolium colchicine products.

In another implementation turfgrass cultivars are developed from the 8 exaploidy (2n=6x=42) progeny resulting from the hybridization of 2n=4x=28 and 2n=8x=56 perennial ryegrass colchicine products.

In another implementation turfgrass cultivars are developed from the 8 exaploidy (2n=6x=42) progeny resulting from the hybridization of 2n=4x=28 Festulolium and the 2n=8x=56 Festulolium colchicine product.

In another implementation turfgrass cultivars are developed from hybridization of 8 exaploidy (2n=6x=42) perennial ryegrass with 8 exaploidy (2n=6x=42) tall fescue.

In another implementation turfgrass cultivars are developed from hybridization of 8 exaploidy (2n=6x=42) Festulolium with 8 exaploidy (2n=6x=42) tall fescue.

### EXAMPLES

Various characteristics of plants can be measured/observed data for the various cultivars is listed in Tables 2-5. In the data, APMT017 refers to the various varieties of American Ryegrass (tetraploid (4x), hexaploid (6x) and octoploid (8x)) and APRT3478 refers to perennial ryegrass (tetraploid (4x), hexaploid (6x) and octoploid (8x)). The measured characteristics may include:
Genetic Color—the measure of the amount of lightness or darkness of green color. Recorded as a 1-9 subjective rating where 9=dark.
Growth Habit—the degree of erectness of a single plant. 1=prostrate (flat), 2=semi-prostrate, 3=horizontal, 7=semi-erect, 9=erect.
Inflorescence—the flowering portion of a grass plant (in ryegrass the inflorescence is a spike).
Spike Length—measured from the upper most node to the apex of the inflorescence.
Node—the joint on a grass culm; a swollen region on the stem.
Leaf Blade—the flattened portion of a grass leaf located above the leaf sheath.
Leaf Blade Length—the length of the leaf blade; measured on the first leaf subtending the flag leaf in cm.
Leaf Blade Width—measure of the width of the first blade subtending the flag leaf in mm taken 1 cm from the collar.
Leaf Blade Height—the height of the leaf blade from the ground to the collar in cm.
Leaf Sheath Length—the length of the leaf sheath; measured on the first leaf subtending the flag leaf in cm.
Flag Leaf—the first leaf blade subtending the inflorescence.
Flag Leaf Length—the total length of a flag leaf which includes the sheath and blade. Measured from the uppermost node to the end of the upper most blade in cm.
Flag Leaf Width—the measure of the width of the flag leaf blade taken 1 cm from the collar of the flag leaf in mm.
Flag Leaf Height—the height of the flag leaf, measured from the ground to the collar of the flag leaf in cm.
Flag Leaf Sheath Length—the sheath length of the flag leaf, measured from the node to the collar in cm.
Mature Plant Height—the height in cm of a mature plant from the ground to the apex of a mature inflorescence.
Glume—the first pair of bracts at the base of a spikelet.
Spikelet—the basic unit of a grass inflorescence, includes glumes, lemmas, paleas and reproductive organs.
Floret—the portion of the spikelet that may include lemma, palea and reproductive organs.
Lemma—an odd veined bract above the glumes.
Palea—the two veined bract above the glumes and lemma, subtending the reproductive organs.
Seed Size—the relative size of seeds usually measured by determining the number of seeds per pound.
1000 seed weight—the weight of 1,000 whole seeds.
Turfgrass Density—the number of tillers per unit area of a turfgrass sward.
Turf Color—a visual and digital analysis score of the turfgrass community, when visual the color is measured on a scale of 1-9 with 9 being dark.
Turf Quality—the degree to which a turf conforms to a standard of uniformity, density, texture, growth habit, color and is generally taken as subjective data on a 1-9 scale with 9 being the best quality.

Although the disclosure has been described with references to various embodiments, persons skilled in the art will recognize that changes may be made in form and detail without departing from the spirit and scope of this disclosure.

What is claimed is:

1. A seed of a hexaploid perennial ryegrass.

2. A grass plant produced by growing the seed of claim 1.

3. A grass plant having all the physiological and morphological characteristics of the grass plant of claim 2.

4. A seed of an octoploid perennial ryegrass.

5. A grass plant produced by growing the seed of claim 4.

6. A grass plant having all the physiological and morphological characteristics of the grass plant of claim 5.

7. A seed of a hexaploid American ryegrass.

8. A grass plant produced by growing the seed of claim 7.

9. A grass plant having all the physiological and morphological characteristics of the grass plant of claim 8.

10. A seed of an octoploid American ryegrass.

11. A grass plant produced by growing the seed of claim 10.

12. A grass plant having all the physiological and morphological characteristics of the grass plant of claim 11.

13. A seed of a hexaploid hybrid of the seed of claim 1 and Festuca arundinacea (Tall Fescue).

14. A grass plant produced by growing the seed of claim 13.

15. A grass plant having all the physiological and morphological characteristics of the grass plant of claim 14.

16. A seed of a hexaploid hybrid of the seed of claim 7 and Festuca arundinacea (Tall Fescue).

17. A grass plant produced by growing the seed of claim 16.

18. A grass plant having all the physiological and morphological characteristics of the grass plant of claim 17.

19. A seed of a hexaploidy hybrid of the seed of claim 13 and the seed of claim 16.

20. A grass plant produced by growing the seed of claim 19.

21. A grass plant having all the physiological and morphological characteristics of the grass plant of claim 20.
